(19) United States
(12) Patent Application Publication
(10) Pub. No.: US 2022/0313438 A1
(43) Pub. Date: Oct. 6, 2022

(54) IMPLANT-ADHERING TECHNIQUES

(71) Applicant: Edwards Lifesciences Innovation (Israel) Ltd., Caesarea (IL)

(72) Inventor: Shlomit Chappel-Ram, Ramat HaSharon (IL)

(21) Appl. No.: 17/843,495

(22) Filed: Jun. 17, 2022

Related U.S. Application Data
(63) Continuation of application No. PCT/IB2020/061251, filed on Nov. 29, 2020.
(60) Provisional application No. 62/951,995, filed on Dec. 20, 2019.

(51) Int. Cl. A61F 2/24; A61B 17/00

(52) U.S. Cl. CPC A61F 2/2466; A61F 2/2445; A61B 17/00491; A61B 2017/00292; A61B 2017/00495; A61B 2017/005; A61F 2220/0008

(57) ABSTRACT

An adhesive-applicator, and an implant that includes a wall formed from a porous fabric, are transcatheterally advanced to a heart of a subject. While a nozzle of the adhesive-applicator is disposed within an interior of the implant, the external surface of the wall is adhered to tissue of the heart by using the adhesive-applicator to apply an adhesive via the nozzle to the interior, such that a portion of the adhesive passes through the porous fabric of the wall to the external surface. Other embodiments are also described.

### CROSS-REFERENCES TO RELATED APPLICATIONS

The present application is a Continuation of International Patent Application PCT/IB2020/061251 to Chappel-Ram, filed Nov. 29, 2020, which published as WO 2021/123975, and which claims priority to U.S. Provisional Patent Application 62/951,995 to Chappel-Ram, filed Dec. 20, 2019, and entitled IMPLANT-ADHERING TECHNIQUES, which is incorporated herein by reference.

### FIELD OF THE INVENTION

Some applications of the present invention relate in general to medical implants. More specifically, some applications of the present invention relate to percutaneous implantation of medical implants.

### BACKGROUND

Dilation of the annulus of a heart valve, such as that caused by ischemic heart disease, prevents the valve leaflets from fully coapting when the valve is closed. Regurgitation of blood from the ventricle into the atrium results in increased total stroke volume and decreased cardiac output, and ultimate weakening of the ventricle secondary to a volume overload and a pressure overload of the atrium.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

For some applications, an adhesive is used to adhere an implant to the tissue. An adhesive-applicator may be used to apply the adhesive to an interior of a porous wall of the implant, such that a portion of the applied adhesive passes through the wall to the external surface of the implant, the portion of adhesive being sufficient to adhere the implant to the tissue.

For some applications, aspects include and/or relate to adhering an annuloplasty structure to an annulus of a heart of a subject. For example, a delivery tool may deploy a sleeve of the annuloplasty structure from a catheter, such that a part of the sleeve meets an adhesion site of the tissue of the annulus. The adhesive-applicator may extend from the catheter, within the sleeve, and adhesive is applied from a nozzle of the adhesive-applicator to the interior of the wall, such that the adhesive passes through the part of the sleeve to the adhesion site. For some such applications, the delivery tool is used to hold the part of the sleeve in place until the adhesive cures into a hardened adhesive.

For some applications, a guide channel is configured to facilitate directed deployment of the implant. At least a distal portion of the guide channel is disposed within the interior of the implant, and at least a portion of the adhesive-applicator is disposed within the guide channel.

For some applications, an adhesive-curing device is configured to cure the adhesive into hardened adhesive, e.g., by applying energy to the adhesive.

For some applications, a first adhesive-component is applied from the adhesive-applicator, and a second adhesive-component may be attached to (e.g., embedded in) the wall.

For some applications, a first adhesive-component is applied from a first nozzle of a first adhesive-applicator, and a second adhesive-component can be applied from a second nozzle of a second adhesive-applicator.

For some applications, the annuloplasty structure includes a contraction member extending along the sleeve of the annuloplasty structure, and an adjustment mechanism of the annuloplasty structure is configured to contract the sleeve by tensioning the contraction member.

There is therefore provided, in accordance with an application, a system and/or an apparatus for use with a tissue of a subject, the system and/or apparatus including a catheter, transluminally advanceable to the tissue, an implant, and an adhesive.

In some applications, the implant is advanceable within the catheter to the tissue and includes at least one porous wall having an external surface and shaped to define an interior.

In some applications, the system and/or apparatus also includes an adhesive-applicator. In some applications, the adhesive-applicator includes a nozzle disposed within the interior and is configured to controllably apply the adhesive to the interior.

In some applications, the adhesive and the wall are configured to allow a portion of the adhesive, when applied to the interior, to pass through the wall to the external surface, the portion of adhesive being sufficient to adhere the implant to the tissue.

In an application, the adhesive includes at least one of lysine-derived urethane and cyanoacrylate.

In an application, the wall includes a polymer.

In an application, the at least one porous wall has an internal surface that faces the interior, and the adhesive-applicator is configured to press the nozzle against the internal surface.

In an application, the nozzle is configured to press against the internal surface.

In an application, the system/apparatus includes a guide channel, at least a distal portion of the guide channel is disposed or can be disposed within the interior.

In an application, at least a portion of the adhesive-applicator is disposed or can be disposed within the guide channel.

In an application, the guide channel is integrated with the adhesive-applicator.

In an application, at least the portion of the adhesive-applicator is axially slidable within the guide channel.

In an application, the wall includes a fabric.

In an application, the wall includes polyethylene terephthalate.

In an application, the adhesive includes a first adhesive-component, the adhesive-applicator is configured to controllably apply the first adhesive-component, and the system/apparatus includes a second adhesive-component.

In an application, at least one adhesive-component selected from the group consisting of the first adhesive-component and the second adhesive-component includes thrombin, and the other adhesive-component of the group includes fibrinogen.

In an application, at least one adhesive-component selected from the group consisting of the first adhesive-component and the second adhesive-component includes albumin, and the other adhesive-component of the group includes glutaraldehyde.

In an application, at least one adhesive-component selected from the group consisting of the first adhesive-component and the second adhesive-component includes gelatin-resorcinol, and the other adhesive-component of the group includes formaldehyde-glutaraldehyde.

In an application, at least one adhesive-component selected from the group consisting of the first adhesive-component and the second adhesive-component includes gelatin-resorcinol, and the other adhesive-component of the group includes pentanedial-ethanedial.

In an application, the second adhesive-component is attached to the wall.

In an application, the system/apparatus includes a second adhesive-applicator, the second adhesive-applicator configured to controllably apply the second adhesive-component.

In an application, the second adhesive-applicator is at least partially disposed within the catheter, includes a second nozzle disposed within the interior, and is configured to controllably apply the second adhesive-component to the interior.

In an application, at least a portion of the second adhesive-applicator is axially slidable within the interior.

In an application, the system/apparatus includes an adhesive-curing device, configured to cure the adhesive by applying energy to the adhesive.

In an application, the adhesive includes a polyethylene hydrogel.

In an application, the adhesive-curing device is configured to apply heat to the adhesive.

In an application, the adhesive-curing device is configured to apply ultraviolet radiation to the adhesive.

In an application, the adhesive includes poly(glycerol sebacate acrylate).

In an application, the adhesive-curing device is disposed within the interior.

In an application, at least a portion of the adhesive-curing device is disposed within the adhesive-applicator.

In an application, at least a portion of the adhesive-curing device is axially slidable within the interior.

In an application, the implant includes an annuloplasty structure, the annuloplasty structure including a sleeve, the porous wall being a tubular lateral wall that defines an elongate lumen through the sleeve.

In an application, the adhesive-applicator is shaped to define a secondary lumen within the elongate lumen.

In an application, the system/apparatus includes a contraction member, the contraction member having a first portion extending along at least a contracting portion of the sleeve and having a second portion exiting the sleeve at an exit point.

In an application, the system/apparatus includes an actuatable adjustment mechanism, the adjustment mechanism: coupled to the contraction member at an end portion of the contraction member, and configured to, when actuated, adjust a length of the annuloplasty structure by applying tension to the contraction member.

In an application, the adjustment mechanism is flexibly connected to the sleeve by a connector.

There is further provided, in accordance with an application, a method for adhering an implant to a tissue of a subject, the method including advancing to the tissue, within a catheter, an implant and an adhesive-applicator (which can be done simultaneously or successively). The adhesive-applicator containing an adhesive.

In an application, the implant includes at least one porous wall having an external surface and shaped to define an interior.

In an application, while a nozzle of the adhesive-applicator is disposed within the interior, the method can include adhering the external surface of the implant to the tissue by using the adhesive-applicator to apply the adhesive via the nozzle to the interior, such that a portion of the applied adhesive passes through the wall to the external surface.

In an application, the method includes contacting the external surface with the tissue prior to using the adhesive-applicator to apply the adhesive via the nozzle into the interior.

In an application, using the adhesive-applicator to apply the adhesive via the nozzle into the interior such that a portion of the applied adhesive passes through the wall to the external surface, includes using the adhesive-applicator to apply the adhesive via the nozzle into the interior such that most of the applied adhesive passes through the wall to the external surface.

In an application, the method includes, while using the adhesive-applicator to apply the adhesive via the nozzle into the interior, pressing the adhesive-applicator against an interior surface of the implant.

In an application, using the adhesive-applicator to apply the adhesive via the nozzle into the interior includes facing the nozzle of the adhesive-applicator flush against an interior surface of the implant.

In an application, the adhesive includes a first adhesive-component, and using the adhesive-applicator to apply the adhesive via the nozzle into the interior includes using the adhesive-applicator to apply the first adhesive-component via the nozzle into the interior such that the first adhesive-component contacts a second adhesive-component and forms a hardened adhesive therewith.

In an application, using the adhesive-applicator to apply the first adhesive-component via the nozzle into the interior includes applying an adhesive-component selected from: thrombin, fibrinogen, albumin, glutaraldehyde, gelatin-resorcinol, formaldehyde-glutaraldehyde and pentanedial-ethanedial.

In an application, the second adhesive-component is attached to the wall, and using the adhesive-applicator to apply the first adhesive-component via the nozzle into the interior applying the first adhesive-component from the adhesive-applicator to the interior such that the first adhesive-component contacts the second adhesive-component and forms the hardened adhesive therewith, includes using the adhesive-applicator to apply the first adhesive-component via the nozzle into the interior such that the first adhesive-component contacts the second adhesive-component attached to the wall and forms the hardened adhesive therewith.

In an application, the method includes using a second adhesive-applicator to apply the second adhesive-component into the interior via a second nozzle of the second adhesive-applicator.

In an application, using the first adhesive-applicator to apply the first adhesive-component via the nozzle into the interior includes applying at least one adhesive-component selected from the group consisting of thrombin and fibrinogen and using the second adhesive-applicator to apply the second adhesive-component via the second nozzle into the interior includes applying the other adhesive-component from the group consisting of thrombin and fibrinogen.

In an application, using the first adhesive-applicator to apply the first adhesive-component via the nozzle into the interior includes applying at least one adhesive-component selected from the group consisting of albumin and glutaraldehyde, and using the second adhesive-applicator to apply the second adhesive-component via the second nozzle into the interior includes applying the other adhesive-component from the group consisting of albumin and glutaraldehyde.

In an application, using the first adhesive-applicator to apply the first adhesive-component via the nozzle into the interior includes applying at least one adhesive-component selected from the group consisting of gelatin-resorcinol and formaldehyde-glutaraldehyde; and using the second adhesive-applicator to apply the second adhesive-component via the second nozzle into the interior includes applying the other adhesive-component from the group consisting of gelatin-resorcinol and formaldehyde-glutaraldehyde.

In an application, using the first adhesive-applicator to apply the first adhesive-component via the nozzle into the interior includes applying at least one adhesive-component selected from the group consisting of gelatin-resorcinol and pentanedial-ethanedial, and using the second adhesive-applicator to apply the second adhesive-component via the second nozzle into the interior includes applying the other adhesive-component from the group consisting of gelatin-resorcinol and pentanedial-ethanedial.

In an application, using the first adhesive-applicator to apply the first adhesive-component via the nozzle into the interior, and using the second adhesive-applicator to apply the second adhesive-component via the second nozzle into the interior, includes applying the first adhesive-component and the second adhesive-component, such that the first adhesive-component and the second adhesive-component pass through the wall and form the hardened adhesive on the external surface.

In an application, the method includes curing the adhesive by applying energy to the adhesive from an adhesive-curing device.

In an application, using the adhesive-applicator to apply the adhesive via the nozzle into the interior includes applying a polyethylene hydrogel into the interior.

In an application, applying energy to the adhesive from the adhesive-curing device includes applying heat to the adhesive from the adhesive-curing device.

In an application, applying energy to the adhesive from the adhesive-curing device includes applying ultraviolet radiation to the adhesive from the adhesive-curing device.

In an application, using the adhesive-applicator to apply the adhesive via the nozzle into the interior includes applying poly(glycerol sebacate acrylate) into the interior.

In an application, the tissue includes tissue of an annulus of a heart of a subject, the implant includes an annuloplasty structure, and adhering the external surface of the implant to the tissue includes adhering an external surface of the annuloplasty structure to the tissue of the annulus.

In an application, the implant includes a sleeve that defines the wall, and using the adhesive-applicator to apply the adhesive via the nozzle into the interior such that the portion of the applied adhesive passes through the wall of the sleeve to the external surface, includes using the adhesive-applicator to apply the adhesive via the nozzle into the interior such that the portion of the applied adhesive passes through the sleeve to the external surface.

In an application, the adhesive-applicator is a component of a delivery tool.

In an application, adhering the external surface of the annuloplasty structure to the tissue of the annulus includes adhering a part of the external surface of the structure to an adhesion site of the tissue of the annulus by deploying the sleeve from the catheter such that the part of the sleeve meets the adhesion site of the tissue of the annulus, extending the adhesive-applicator from the catheter, within the sleeve, and applying the adhesive from the nozzle of the adhesive-applicator to the interior of the implant, such that the adhesive passes through the part of the sleeve to the adhesion site. In an application, the delivery tool is used or is usable to hold the part of the sleeve in place until the adhesive cures into a hardened adhesive.

In an application, using the delivery tool to hold the part in place includes using the adhesive-applicator to hold the part in place.

In an application, the method includes detaching the structure from the delivery tool, and transluminally retracting the delivery tool from the heart of the subject.

In an application, the part of the external surface is a first part of the external surface, the adhesion site is a first adhesion site, adhering the part of the external surface of the wall to the adhesion site includes adhering the first part of the external surface of the wall to the first adhesion site of the tissue of the annulus, and the method includes, subsequently to adhering the first part to the first adhesion site, adhering a second part of the external surface of the wall to a second adhesion site of the tissue of the annulus by applying the adhesive from the nozzle of the adhesive-applicator to the interior of the implant, such that the adhesive passes through the second part of the sleeve to the second adhesion site.

In an application, the first adhesion site is at a left fibrous trigone, and adhering the first part of the external surface to the first adhesion site includes adhering the first part of the external surface to the first adhesion site at the left fibrous trigone of the annulus.

In an application, the first adhesion site is at a right fibrous trigone, and adhering the first part of the external surface to the first adhesion site includes adhering the first part of the external surface to the first adhesion site at the right fibrous trigone of the annulus.

In an application, the annuloplasty structure includes a contracting portion, the structure includes a contraction member extending along at least the contracting portion of the sleeve, and the method includes contracting the contracting portion by tensioning the contraction member.

In an application, contracting the contracting portion by tensioning the contraction member includes adjusting an adjustment mechanism.

In an application, the adjustment mechanism includes a rotatable spool, the spool coupled to the contraction member, and contracting the contracting portion by tensioning the contraction member includes rotating the spool.

In an application, contracting the contracting portion by tensioning the contraction member includes adjusting a perimeter of the sleeve.

In an application, adjusting the perimeter of the sleeve includes shortening an inter-adhesion-location distance.

In an application, adjusting the perimeter of the sleeve includes shortening an inter-adhesion site distance.

These methods can be performed on a living animal or on a simulation, such as on a cadaver, cadaver heart, simulator (e.g. with the body parts, heart, tissue, etc. being simulated), etc.

There is further provided, in accordance with an application, a system and/or an apparatus for use with a tissue of a subject, the system and/or apparatus including a catheter that is transluminally advanceable to the tissue, an implant, an adhesive, and an adhesive applicator.

In some applications, the implant is advanceable within the catheter to the tissue, includes at least one porous wall, and is shaped to define an interior.

In some applications, the adhesive-applicator has a nozzle disposed within the interior and is configured to controllably apply the adhesive into the interior.

In some applications, the adhesive and the wall are configured such that, while the wall is disposed against the tissue, applying the adhesive to the interior via the nozzle causes at least a portion of the applied adhesive to pass through the wall and adhere the implant to the tissue.

The present invention will be more fully understood from the following detailed description of applications thereof, taken together with the drawings, in which:

### BRIEF DESCRIPTION OF THE DRAWINGS

FIG. 1 is a schematic illustration of a multi-component system comprising an implant and a delivery tool for delivering the implant to a heart of a subject, in accordance with some applications;

FIGS. 2A-B are schematic illustrations showing the implant and the tool being deployed to an annulus of the heart, in accordance with some applications;

FIGS. 3A-F are schematic illustrations showing an adhesive being applied from an adhesive-applicator to an interior of a wall of the implant, in accordance with some applications;

FIGS. 4A-E are schematic illustrations showing the adhesive being applied from the adhesive-applicator, and energy being applied from an adhesive-curing device to the adhesive, in accordance with some applications;

FIGS. 5A-E are schematic illustrations showing a multi-component system comprising an implant and a delivery tool for delivering the implant to the heart of a subject, in accordance with some applications; and

FIGS. 6A-E are schematic illustrations showing a multi-component system comprising an implant and a delivery tool for delivering the implant to the heart of a subject, in accordance with some applications.

### DETAILED DESCRIPTION OF EMBODIMENTS

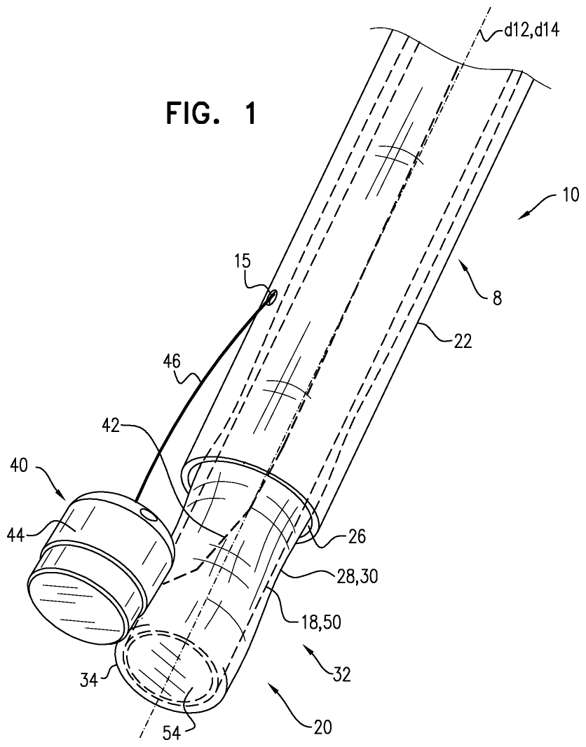

Reference is made to FIG. 1, which is a schematic illustration of a multi-component system 10 comprising an implant, and a delivery tool 8 for delivering the implant to a heart 90 of a subject, in accordance with some applications. Throughout this application, the implant of system 10 is described as embodied as an annuloplasty structure 20. However, it is to be noted that, for some applications, the systems, apparatuses, and techniques described herein may be used to facilitate implantation of other implants, mutatis mutandis.

FIG. 1 shows a distal portion of system 10 comprising annuloplasty structure 20 (e.g., an annuloplasty band), disposed partially within guide a catheter 22 of tool 8. Sleeve 30 is typically a flexible sleeve comprising a braided fabric mesh, e.g., comprising polyethylene terephthalate (such as Dacron™). Sleeve 30 is typically configured to be placed only partially around a cardiac valve annulus 88 (i.e., to assume a C-shape), and, once adhered to the annulus, to be contracted so as to adjust a perimeter of the annulus (i.e., to circumferentially tighten the annulus). Alternatively, structure 20 is configured to be placed entirely around annulus 88.

Typically, and as shown, structure 20 defines an interior, and sleeve 30 comprises a tubular lateral wall 28 having an external surface. For some applications, and as shown, sleeve 30 defines an elongate lumen (e.g., the interior of structure 20 is shaped as an elongate lumen). For some applications, an end wall 34 defines an end wall of annuloplasty structure 20.

Delivery tool 8 further comprises an adhesive-applicator 50 configured to controllably apply an adhesive 52 to the interior of structure 20. For some applications, distal segment 32 of adhesive-applicator 50 comprises or defines a nozzle 54, which facilitates controlled application of adhesive 52 to the interior of structure 20. For example, a nozzle 54 of the adhesive-applicator may be disposed within (or may be advanceable into) the interior of structure 20. For some applications, adhesive-applicator 50 is shaped to define a secondary lumen within the elongate lumen of sleeve 30.

For some applications, tool 8 comprises a guide channel 18 configured to facilitate directed deployment of structure 20, e.g., as described, mutatis mutandis, in US Patent Application Publication 2018/0049875 to Iflah et al., which is incorporated herein by reference. For such applications, at least a distal portion of guide channel 18 is disposed within the interior of structure 20. For some applications, sleeve 30 comprises a flexible material, such that the sleeve is moved (e.g., advanced) into position by moving guide channel 18. For some such applications, a portion of adhesive-applicator 50 is disposed within guide channel 18. For some such applications, at least a portion of adhesive-applicator 50 is axially slidable within guide channel 18. For some such applications, a longitudinal axis d12 of adhesive-applicator 50 is generally parallel to a longitudinal axis d14 of guide channel 18.

For some applications, and as shown in FIG. 1, adhesive-applicator 50 is integrated with (e.g., defined by) guide channel 18.

For some applications, soon before implantation (e.g., within the operating theater or in an adjacent room) the distal portion of channel 18 is loaded into sleeve 30, and structure 20 is loaded into catheter 22.

For some applications, annuloplasty structure 20 comprises a flexible elongated contraction member 42 that extends along at least a portion of sleeve 30, the portion of the sleeve along which member 42 extends thereby being defined as a contracting portion of the sleeve. Typically, a first portion of contraction member 42 extends along the contracting portion of sleeve 30, and a second portion of the contraction member exits the sleeve at an exit point. Contraction member 42 may comprise a wire, a ribbon, a rope, or a band, and typically comprises a flexible and/or superelastic material, e.g., nitinol, polyester, stainless steel, or cobalt chrome. For some applications, the wire comprises a radiopaque material. For some applications, contraction member 42 comprises a braided polyester suture (e.g., Ticron). For some applications, contraction member 42 is coated with polytetrafluoroethylene (PTFE). For some applications, contraction member 42 comprises a plurality of wires that are intertwined to form a rope structure.

For some applications, annuloplasty structure 20 further comprises an adjustment mechanism 40, which facilitates contracting and expanding of annuloplasty structure 20. Adjustment mechanism 40 may be disposed within a housing 44 and may comprise a rotatable structure (e.g., a spool, as described hereinbelow). Adjustment mechanism 40 is coupled to contraction member 42 at an end portion of the contraction member. When actuated, adjustment mechanism 40 adjusts a length of structure 20 by applying tension to contraction member 42. Adjustment mechanism 40 may be coupled (e.g., by being sutured or otherwise coupled) to sleeve 30. For some applications, adjustment mechanism 40 is coupled to an outer, lateral surface of sleeve 30.

For some applications in which annuloplasty structure 20 comprises adjustment mechanism 40, system 10 comprises a flexible, longitudinal guide member 46 (e.g., a wire) coupled to a portion of the adjustment mechanism (e.g., a portion of the rotatable structure). Guide member 46 extends from adjustment mechanism 40 and proximally through catheter 22 (e.g., through a parallel side-lumen of the catheter) and has a proximal end that is accessible from outside the body of the subject.

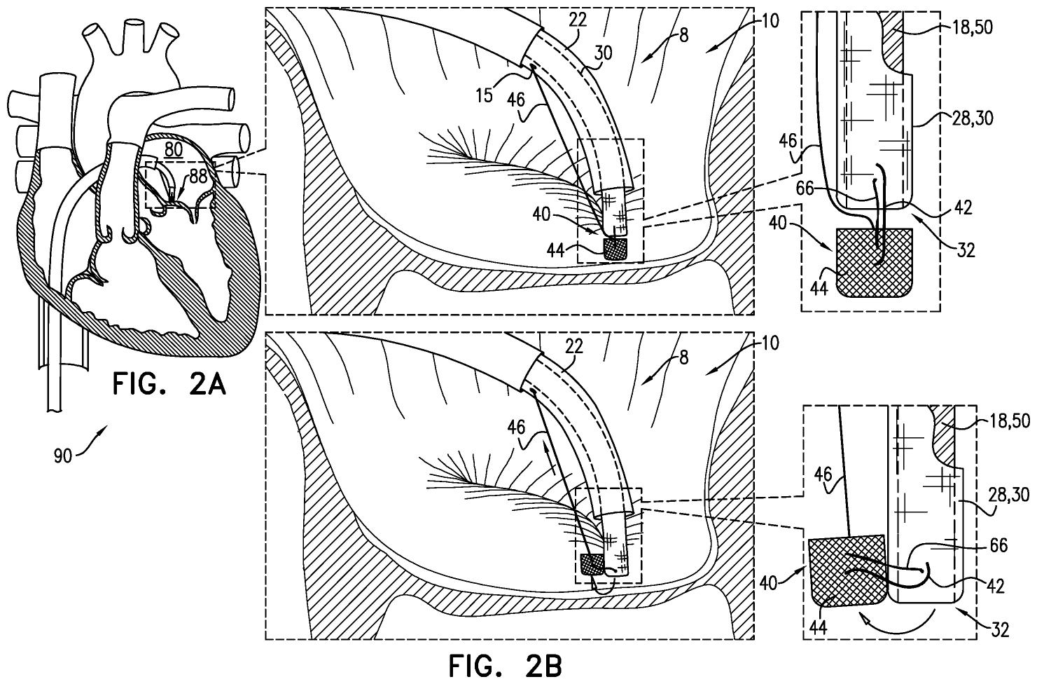

Reference is made to FIGS. 2A-B, which are schematic illustrations showing structure 20 and tool 8 being deployed to an annulus 88 of heart 90, in accordance with some applications.

Annuloplasty structure 20 is advanced into left atrium 80 using catheter 22 (FIG. 2A). For some applications, and as shown, this is performed by advancing catheter 22 with annuloplasty structure 20 disposed therein. Alternatively, catheter 22 may be advanced first, and annuloplasty structure 20 (or another implant) may be subsequently advanced through the catheter. For some applications, and as shown, annuloplasty structure 20 may be advanced with the distal portion of channel 18 and/or adhesive-applicator 50 (e.g., nozzle 54) disposed in the interior of the annuloplasty structure. Alternatively, channel 18 and/or adhesive-applicator 50 (e.g., nozzle 54) may be introduced into the interior after advancement of annuloplasty structure 20 (or another implant). While a transfemoral transseptal approach to the mitral valve is shown in FIG. 2A, the scope herein includes alternate approaches to the mitral valve, to other locations in (e.g., valves of) the heart, and to other locations in the body.

For some applications in which annuloplasty structure 20 comprises adjustment mechanism 40, the adjustment mechanism is disposed distal to (i.e., in front of) the structure during advancement of the structure. For example, adjustment mechanism 40 may be disposed on axis d12 (e.g., collinearly with sleeve 30). For some such applications, mechanism 40 is coupled to sleeve 30 in a manner that allows mechanism 40 to move (e.g., to translate) from a state in which it is in line with axis d12, to a state in which it is disposed alongside sleeve 30 (FIG. 2B). For some applications it is advantageous to (1) advance the structure to the mitral valve while mechanism 40 is disposed on the longitudinal axis of sleeve 30 (e.g., collinearly with the sleeve), so as to maintain a small cross-sectional diameter of the structure for transluminal delivery; and (2) to subsequently move mechanism 40 away from the longitudinal axis, e.g., so as to allow end wall 34 of the sleeve to be placed against the annulus, and/or so as to allow adhesive to be applied through the end wall of the sleeve.

For some applications, one or more connectors 66 (e.g. sutures) facilitate translation of adjustment mechanism 40 by flexibly and/or articulatably coupling the mechanism to sleeve 30. For some such applications, connectors 66 are tensioned or relaxed to reposition mechanism 40 with respect to sleeve 30. For some applications, guide member 46 is tensioned or relaxed in order to reposition mechanism 40.

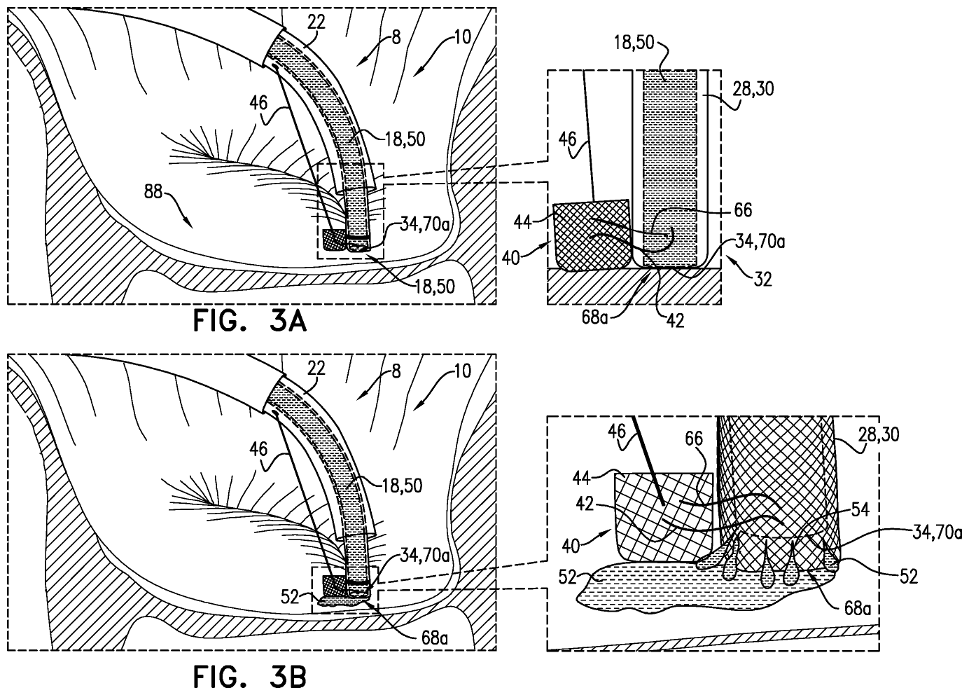

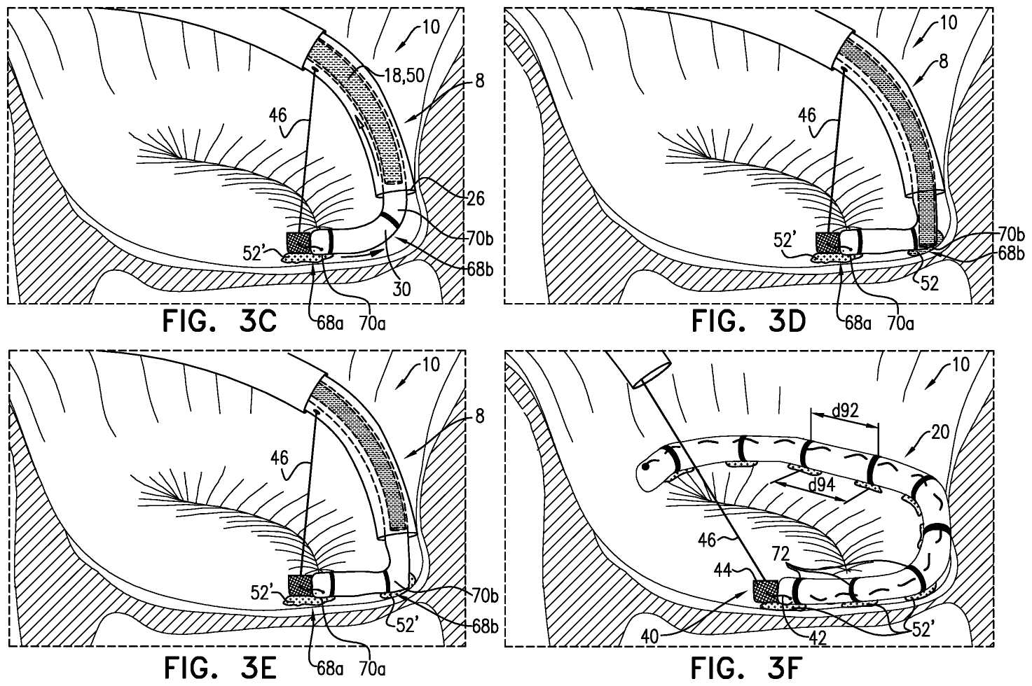

Reference is made to FIGS. 3A-E, which are schematic illustrations showing structure 20 being adhered to tissue by applying an adhesive 52 from adhesive-applicator 50 to the interior of structure 20, in accordance with some applications.

In FIG. 3A, guide channel 18 is shown abutting end wall 34 of sleeve 30, such that steering of the guide channel directs the end wall to a first adhesion site 68a. Sleeve 30 typically comprises a flexible material, such as a fabric (e.g. polyethylene terephthalate), such that the sleeve is advanced into position by use of guide channel 18. That is, guide channel 18 is typically more rigid than sleeve 30, such that steering of the guide channel effectively steers the sleeve. As described hereinabove, guide channel 18 may be a discrete element within which adhesive-applicator 50 is disposed, or adhesive-applicator 50 may be integrated with, or even serve as, guide channel 18.

As shown in FIGS. 3A-E, during implantation of annuloplasty structure 20, adhesive-applicator 50 is disposed within the interior of the annuloplasty structure and is used to controllably apply an adhesive 52 to the interior of the annuloplasty structure. At least part of wall 28 is porous, and the wall and the adhesive are configured to allow a portion of the adhesive, when applied to the interior, to pass through the wall to the external surface of the wall, where it adheres the implant to the tissue.

For some applications, and as shown in FIG. 3A, the first site of the tissue to which structure 20 is adhered, is in a vicinity of a left fibrous trigone 82 of annulus 88. Alternatively, the first site is in a vicinity of a right fibrous trigone of the mitral valve (not shown). Further alternatively, end wall 34 is not positioned in the vicinity of either of the trigones, but is instead positioned elsewhere in a vicinity of the mitral valve, such as in a vicinity of the anterior or posterior commissure (not shown).

For some applications, and as shown, structure 20 is adhered to the tissue by adhering a plurality of discrete parts 70 of the structure to a corresponding plurality of discrete adhesion sites 68 of the tissue, e.g., by applying a corresponding plurality of discrete portions of adhesive 52 at the corresponding plurality of parts of the structure. (This may be the case, irrespective of whether structure 20 has particularly defined features that define parts 70. That is, parts 70 may be defined as discrete parts of structure 20 by virtue of the application of the discrete portions of adhesive 52.) Alternatively, adhesive 52 may be applied throughout the interior of structure 20.

For some applications, and as shown, the first part of structure 20 to be adhered to the tissue (e.g., to first adhesion site 68a) is distal end wall 34 of sleeve 30. That is, for some applications, distal end wall 34 defines a first part 70a of the structure to be adhered to the tissue.

As described hereinabove, and as shown in FIG. 3B, adhesive 52 is applied from nozzle 54 of adhesive-applicator 50 to the interior of structure 20 (e.g. interior of wall 28) such that at least some of the adhesive passes through the wall, at part 70a, to the external surface of wall 28. Typically, the external surface of wall 28 is contacted to the tissue (e.g. adhesion site 68a of annulus 88) prior to applying adhesive 52 into the interior. Alternatively, the adhesive may be applied into the interior prior to contacting the wall to the tissue. In this way, the external surface of structure 20 is adhered to annulus 88.

For some such applications, nozzle 54 of adhesive-applicator 50 is pressed against the interior of wall 28 while adhesive is applied from the adhesive-applicator. For some applications, nozzle 54 meets flush with wall 28 while adhesive 52 is applied at first part 70a.

At least a portion (e.g., most) of adhesive 52 that is applied via nozzle 54 into the interior of structure 20 passes through wall 28 to the external surface of the structure. For some such applications, the adhesive is directed to pass through wall 28 toward adhesion site 68. It is hypothesized by the inventors that flush meeting of nozzle 54 with the internal surface of wall 28, and/or pressing adhesive-applicator 50 to the internal surface, while applying adhesive 52, facilitates: directing of the adhesive through the wall to adhesion site 68, and/or adhesion of part 70 to the adhesion site, e.g., by reducing leaking of the adhesive laterally of the nozzle.

Typically, adhesive 52, within adhesive-applicator 50, is in a fluidic state. As shown in inset of FIG. 3B, porous wall 28 is configured to allow adhesive 52 to pass through the wall. Typically, the portion of adhesive 52 that passes through wall 28 is sufficient to adhere structure 20 (e.g., part 70 thereof) to the tissue (e.g., adhesion site 68 thereof). Passage of sufficient adhesive 52 through wall 28 is typically facilitated by at least one of the following factors: (i) porosity of wall 28, (ii) fluidity of adhesive 52, and (iii) interaction between the wall and the adhesive (e.g., surface phenomena).

It is hypothesized by the inventors that, for some applications, adhering structure 20 to the tissue by applying adhesive 52 to the interior of the structure, may more readily facilitate proper placement of the structure, relative to the tissue, compared to if the adhesive were applied directly to the tissue prior to placing the structure. For example, application of adhesive 52 after placement of structure 20 may (1) afford the operator with additional time during which to steer guide catheter 22 and/or guide channel 18; and (2) may enable repositioning of the structure, in the event that the initial position may appear to be sub-optimal, prior to application of adhesive 52.

Typically, after passing through wall 28, and while adhesive 52 maintains contact with both wall 28 and the tissue, the adhesive undergoes curing. As is known in the art, curing is a process involving hardening and/or strengthening of an adhesive. For some applications, delivery tool 8 (e.g. adhesive-applicator 50 and/or guide channel 18) is used to hold part 70 in place until adhesive 52 cures into a hardened adhesive 52′. As shown in FIG. 3C, hardened adhesive 52′ adheres first part 70a of sleeve 30 to first adhesion site 68a of annulus 88.

For some applications, the rate at which adhesive 52 undergoes curing occurs may depend upon application of energy (e.g. in the form of heat or light). In other applications, adhesive 52 may undergo curing independently of applied energy. For some applications, adhesive 52 comprises cyanoacrylate. For some applications, adhesive 52 comprises lysine-derived urethane. For some applications, adhesive 52 comprises polyethylene hydrogel. For some applications, adhesive 52 comprises poly(glycerol sebacate acrylate). This list is not meant to be exhaustive, and the scope herein includes use of other suitable adhesives.

Following adhesion of first part 70a to first adhesion site 68a, a portion of structure 20 (e.g. sleeve 30) is typically advanced off of guide channel 18, e.g., as described in US Patent Application Publication 2018/0049875 to Iflah et al., mutatis mutandis (FIG. 3C). After the portion of sleeve 30 is freed in this way, a second part 70b of structure 20 is positioned at a second adhesion site 68b, e.g., by repositioning channel 18, adhesive-applicator 50, and or catheter 22 (FIGS. 3C-D).

As shown in FIG. 3D, while second part 70b is aligned to second adhesion site 68b, adhesive 52 is again applied from adhesive-applicator 50, through porous wall 28 at second part 70b, to second adhesion site 68b. As shown in FIG. 3E, adhesive 52 then undergoes curing, such that hardened adhesive 52′ adheres second part 70b to second adhesion site 68b. Typically, application and curing of adhesive is repeated at a plurality of respective parts 70 and adhesion sites 68. As shown in FIG. 3F, this repetition results in adhesion of successive parts 70 to successive adhesion sites 68.

Parts 70 may be longitudinally spaced at a constant interval along sleeve 30 or may be spaced as deemed suitable by the operating physician.

For some applications, sleeve 30 comprises a plurality of radiopaque markers 72, which are positioned along the sleeve at respective longitudinal sites. For some applications, markers 72 comprise a radiopaque ink. Markers 72 may provide an indication in a radiographic image (such as a fluoroscopy image) of how much of the sleeve has been deployed at any given point during an implantation procedure, e.g., in order to enable setting a desired distance between adhered parts 70 along the sleeve.

For some applications, the longitudinal distance between adjacent/consecutive adhered parts 70 is approximately equal to the longitudinal distance between adjacent/consecutive markers 72. For example, and as shown, a portion of adhesive 52 may be applied approximately at each of markers 72. Alternatively or additionally, a portion of adhesive 52 may be applied between adjacent/consecutive markers.

Typically, after structure 20 is adhered to annulus 88, sleeve 30 (e.g., the contracting portion thereof) is contracted, e.g., using adjustment mechanism 40. For example, an adjustment tool may be advanced along (e.g., over and along) guide member 46 to adjustment mechanism 40 and may be used to actuate the adjustment mechanism. For some such applications, adjustment mechanism 40 is configured to adjust a perimeter of annuloplasty structure 20 by tensioning contraction member 42, e.g., as described in US Patent Application Publication 2018/0049875 to Iflah et al., mutatis mutandis.

Due to the adherence of parts 70 to adhesion sites 68, adjusting (e.g., reducing) the perimeter of annuloplasty structure 20 adjusts (e.g., reduces) the perimeter of annulus 88. For example, this may shorten: (i) inter-part distances d92 between adhered parts 70 of structure 20, and (ii) inter-adhesion site distances d94 between adhesion sites 68 of the annulus.

It is hypothesized by the inventors that, for some applications, adhering annuloplasty structure 20 only at discrete parts of the annuloplasty structure (e.g., rather than applying adhesive throughout the annuloplasty structure) may facilitate contraction of the annuloplasty structure and the annulus, e.g., due to non-adhered parts of the annuloplasty structure offering less resistance to contraction compared to adhered parts of the annuloplasty structure.

Once the desired level of adjustment of structure 20 is detected, e.g., by monitoring the extent of regurgitation of the valve using echocardiography (such as Doppler echocardiography) and/or fluoroscopy, the adjustment tool and guide member 46 are removed from the heart. Typically, annuloplasty structure 20 is detached from delivery tool 8, and the delivery tool is transluminally retracted from heart 90 of the subject.

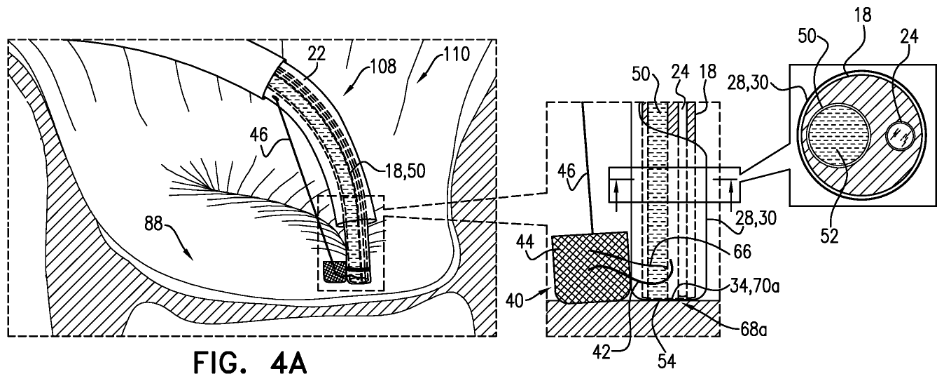

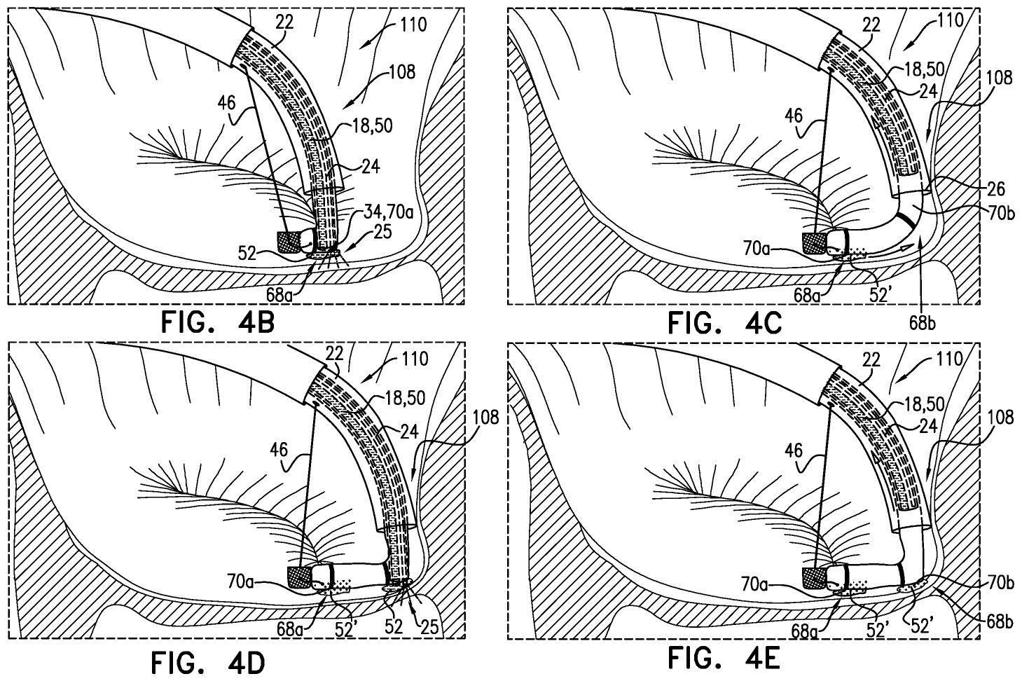

Reference is made to FIGS. 4A-E, which are schematic illustrations of a multi-component system 110 comprising an implant (e.g., annuloplasty structure 20), and a delivery tool 108 for delivering the implant to a heart 90 of a subject, in accordance with some applications. Except where noted, system 110 and tool 108 are typically identical to system 10 and tool 8, mutatis mutandis. Similarly, the techniques shown in FIGS. 4A-E are typically identical to system 10 and tool 8 except where noted, mutatis mutandis. Tool 108, in addition to the components of tool 8, also comprises an adhesive-curing device 24.

Adhesive-curing device 24 is configured to cure adhesive 52 into hardened adhesive 52′ by applying energy 25 to the adhesive. Typically, and as shown, adhesive-curing device 24 is disposed within the interior of structure 20 (e.g. within sleeve 30). For some applications, adhesive-curing device 24 is disposed within guide channel 18. For some applications, adhesive-curing device 24 is a component of adhesive-applicator 50. Further typically, at least a portion of adhesive-curing device 24 is axially slidable within the interior of structure 20. For some applications, adhesive-curing device 24 is maneuverable independently of adhesive-applicator 50. As shown in FIG. 4B, adhesive-applicator 50 applies adhesive 52, which passes through wall 28 to the external surface of the implant (e.g. through first part 70a to first adhesion site 68a), e.g., as described hereinabove with reference to system 10, mutatis mutandis. Adhesive-curing device 24 applies energy 25 to applied adhesive 52, curing the adhesive into hardened adhesive 52′.

Adhesive-curing device 24 may be configured to apply a variety of forms of energy for curing adhesive that are known in the art, including but not limited to electromagnetic radiation (e.g., ultraviolet or infrared light), heat, and/or acoustic energy (e.g., ultrasound). For some applications, adhesive-curing device 24 transmits energy provided by an external energy source. For example, adhesive-curing device 24 may comprise a channel configured to transmit energy (e.g., an optical fiber). For some applications, adhesive 52 comprises polyethylene hydrogels. For some applications, adhesive 52 comprises poly(glycerol sebacate acrylate). This list is not meant to be exhaustive, and the scope herein includes use of other adhesives.

As shown in FIGS. 4C-E, tool 108 may be used to adhere a plurality of discrete parts 70 of structure 20 to a corresponding plurality of adhesion sites 68, e.g., as described with reference to FIGS. 3C-E, mutatis mutandis. Subsequent contraction of annuloplasty structure 20, detachment of the structure from delivery tool 108, and transluminal retraction of the tool, are described hereinabove, mutatis mutandis.

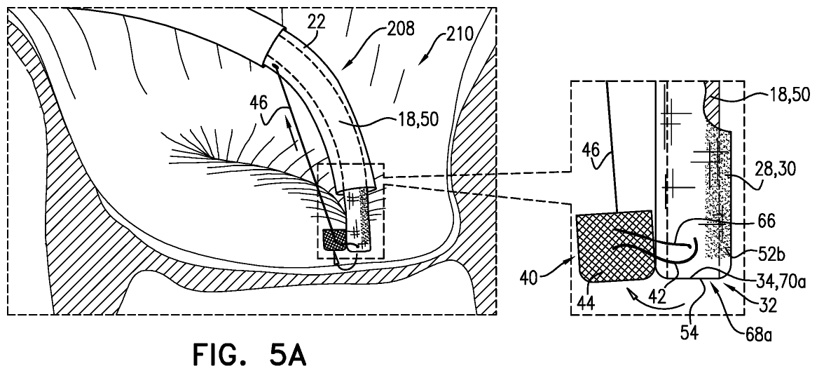

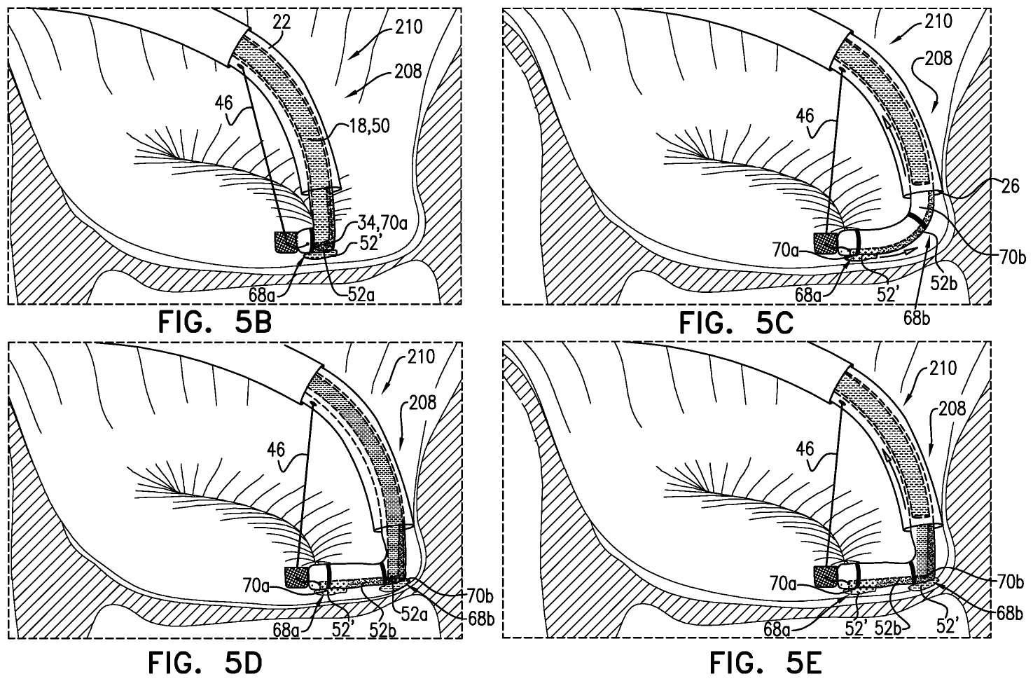

Reference is now made to FIGS. 5A-E and 6A-E, which are schematic illustrations of respective multi-component systems 210 and 310, each system comprising an implant (e.g., annuloplasty structure 20), and a respective delivery tool 208 and 308 for delivering the implant to a heart 90 of a subject, in accordance with some applications.

In systems 210 and 310, a first adhesive-component 52a and a second adhesive-component 52b are used to adhere the implant to tissue of annulus 88. Typically, adhesive-components 52a and 52b are combined (e.g., contacted and/or mixed) with each other at the implant to be adhered. For some applications, combined adhesive-components 52a and 52b may be considered to be adhesive 52. Typically, after they are combined, first adhesive-component 52a and second adhesive-component 52b cure into hardened adhesive 52′. For some applications, formation of hardened adhesive 52′ by first adhesive-component 52a and second adhesive-component 52b may obviate the use of adhesive-curing device 24.

Alternatively, tool 208 may comprise adhesive-curing device 24, and adhesive-curing device 24 is used complementarily with first adhesive-component 52a and second adhesive-component 52b, to yield hardened adhesive 52′ (not shown). That is, as described hereinabove regarding FIG. 4A, curing of first adhesive-component 52a and second adhesive-component 52b into hardened adhesive 52′ may involve application of energy (e.g., electromagnetic radiation, heat, and/or acoustic energy). For some applications, one of adhesive-components 52a and 52b comprises thrombin, and the other comprises fibrinogen. For some applications, one of adhesive-components 52a and 52b comprises albumin, and the other comprises glutaraldehyde. For some applications, one of adhesive-components 52a and 52b comprises gelatin-resorcinol, and the other comprises an aliphatic dialdehyde (e.g. pentanedial and/or ethanedial). For some applications, one of adhesive-components 52a and 52b comprises gelatin-resorcinol, and the other comprises formaldehyde. This list is not meant to be exhaustive, and the scope herein includes use of other adhesives.

Reference is again made to FIGS. 5A-E, which are schematic illustrations showing use of tool 208 to deliver the implant to a heart 90 of a subject, in accordance with some applications.

Except where noted, system 210 and tool 208 are typically identical to system 10 and tool 8, mutatis mutandis. Similarly, the techniques shown in FIGS. 5A-E are typically identical to system 10 and tool 8 except where noted, mutatis mutandis. Tool 208 is essentially similar to tool 8, but rather than applying adhesive 52, it applies first adhesive-component 52a from adhesive-applicator 50. Second adhesive-component 52b is typically already present outside of tool 208.

Typically, and as shown in FIG. 5A, second adhesive-component 52b is attached to wall 28. For example, second adhesive-component 52b may be coated on and/or embedded within wall 28.

First adhesive-component 52a is typically applied from adhesive-applicator 50 to the interior of structure 20, while sleeve 30 is already disposed within the body of the subject, e.g., in the desired anatomical location (FIG. 5B). As described hereinabove, porosity of wall 28 facilitates passage of first adhesive-component 52a through the wall to the external surface of the implant. Typically, adhesive-applicator 50 is used to apply first adhesive-component 52a via nozzle 54 to the interior, such that the first adhesive-component contacts the second adhesive-component attached to wall 28, and forms hardened adhesive 52′ therewith. In this way, first adhesive-component 52a and second adhesive-component 52b form hardened adhesive 52′ on the external surface of sleeve 30, adhering structure 20 to tissue of annulus 88.

As shown in FIGS. 5C-E, tool 208 may be used to adhere a plurality of discrete parts 70 of structure 20 to a corresponding plurality of discrete adhesion sites 68, e.g., as described with reference to FIGS. 3C-E, mutatis mutandis. For some applications, adhesive-component 52b is disposed only at pre-determined parts 70 of structure 20. For some applications, and as shown, adhesive-component 52b is disposed more broadly along structure 20 (e.g., along all of structure 20) such that parts 70 to be adhered are defined by the application of adhesive-component 52a, e.g., such that portions of adhesive-component 52b disposed between parts 70 are left unused.

Subsequent contraction of annuloplasty structure 20, detachment of the structure from delivery tool 208, and transluminal retraction of the tool, are described hereinabove, mutatis mutandis.

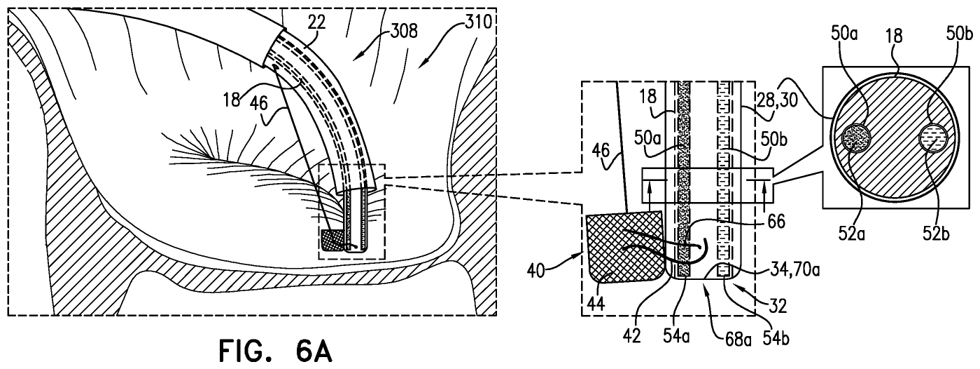

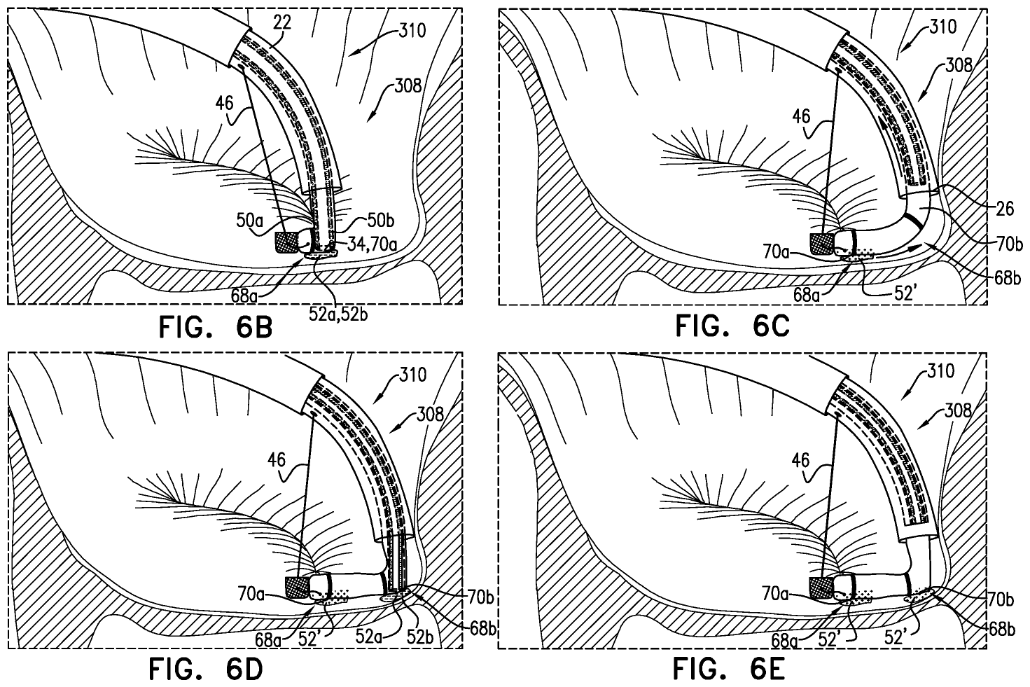

Reference is again made to FIGS. 6A-E, which are schematic illustrations showing use of tool 308 to deliver the implant to a heart 90 of a subject, in accordance with some applications.

Except where noted, system 310 and tool 308 are typically identical to system 10 and tool 8, mutatis mutandis. Similarly, the techniques shown in FIGS. 6A-E are typically identical to system 10 and tool 8 except where noted, mutatis mutandis. Tool 308 is essentially similar to tool 8, but rather than comprising adhesive-applicator 50, tool 308 comprises a first adhesive-applicator 50a and a second adhesive-applicator 50b. First adhesive-applicator 50a is configured to controllably apply first adhesive-component 52a to the interior of the implant, and second adhesive-applicator 50b is configured to controllably apply second adhesive-component 52b to the interior of the implant. For some applications, adhesive-applicators 50a and 50b contain their respective adhesive-component, e.g., prior to advancement of tool 308 into the subject.

Typically, first adhesive-applicator 50a and second adhesive-applicator 50b are each at least partially disposed within catheter 22. Further typically, controlled application of adhesive is facilitated by each adhesive-applicator comprising a respective nozzle. As shown in FIGS. 6A-B, first adhesive-applicator 50a is used to apply first adhesive-component 52a via a first nozzle 54a, and second adhesive-applicator 50b is used to apply second adhesive-component 52b via a second nozzle 54b. For some applications, a portion (e.g. a first nozzle 54a) of first adhesive-applicator 50a and a portion (e.g. a second nozzle 54b) of second adhesive-applicator 50b are axially slidable within the interior of structure 20.

Typically, first adhesive-component 52a and second adhesive-component 52b pass through wall 28 and cure to form hardened adhesive 52′ on the external surface of the structure. As described hereinabove regarding adhesive 52, respective adhesive components 52a and 52b each typically retain a fluidic state when kept separate from the other adhesive-component. For some applications, contact between the adhesive-components is typically sufficient to yield hardened adhesive 52′. In other applications (not shown), tool 308 comprises adhesive-curing device 24, and application of energy by the adhesive-curing device accelerates curing of the respective adhesive components into hardened adhesive 52′.

As shown in FIGS. 6C-E, tool 308 may be used to adhere a plurality of discrete parts 70 of structure 20 to a corresponding plurality of discrete adhesion sites 68, e.g., as described with reference to FIGS. 3C-E, mutatis mutandis. Alternatively, adhesive-components 52a and 52b may be applied throughout the interior of structure 20. Subsequent contraction of annuloplasty structure 20, detachment of the structure from delivery tool 308, and transluminal retraction of the tool, are described hereinabove, mutatis mutandis.

Curable compositions suitable for use as adhesives in connection with the implants described herein can comprise a crosslinking pre-polymer and an initiator. Exemplary curable compositions that can be used in connection with the implantable medical devices disclosed herein are described in PCT Publication No. WO 2018/175619, published Sep. 27, 2018, and U.S. Patent Application Publication No. 2014/0348896, published Nov. 27, 2014, the entire contents of which are incorporated herein by reference. In a preferred embodiment, the pre-polymer comprises one or more of the following characteristics: (1) the pre-polymer has a sufficient viscosity such that it withstands the hemodynamic forces and resists being washed off the site of application; (2) the pre-polymer is not reactive with or does not crosslink in the presence of bodily fluids and, in particular, blood; (3) the pre-polymer is hydrophobic; (4) the pre-polymer is capable of adhering to wet tissue; (5) the pre-polymer is biocompatible; and (6) the pre-polymer is biodegradable.

In one application, the pre-polymer is activated by introduction of one or more functional groups (i.e., incorporated on the pre-polymer backbone) that can be reacted to form crosslinks between polymer chains. In one embodiment, the functional groups can be selected from the group consisting of: substituted vinyl groups, unsubstituted vinyl groups, substituted acrylate groups, unsubstituted acrylate groups, vinyl esters, vinyl carbamates, vinyl ketones, vinyl amides, vinyl carbonates, vinyl ether groups or vinyl groups in the form of allyl. In one embodiment, the polymer chain is polyester formed from a substituted or unsubstituted polyol, such as a triol, and a substituted or unsubstituted diacid. The triol can be glycerol. The functional groups can also form crosslinks with the tissue. The degree of activation can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, or 1.5. The degree of activation can be provided within a range of between and including any two of the foregoing values.

The degree of activation can be selected based on whether the curable composition is a sealant or an adhesive. Generally, the degree of activation for a sealant is expected to be lower than the degree of activation for an adhesive.

In one application, the curable composition comprises or consists of a sealant and the pre-polymer has a degree of activation that is about 0.5 or less, about 0.4 or less, about 0.3 or less, about 0.2 or less, about 0.1 or less, about 0.09 or less, about 0.08 or less, about 0.07 or less, about 0.06 or less, about 0.05 or less, about 0.04 or less, about 0.03 or less, about 0.02 or less, about 0.01 or less, about 0.009 or less, about 0.008 or less, about 0.007 or less, about 0.006 or less, about 0.005 or less, about 0.004 or less, about 0.003 or less, about 0.002 or less, or about 0.001 or less.

In one application, the curable composition comprises or consists of an adhesive and the pre-polymer as a degree of activation that is about 0.5 or greater, 0.6 or greater, 0.7 or greater, 0.8 or greater, 0.9 or greater, 0.1 or greater, 0.2 or greater, 0.3 or greater, 0.4 or greater, 0.5 or greater, 0.6 or greater, 0.7 or greater, 0.8 or greater, 0.9 or greater, 1.0 or greater, 1.1 or greater, 1.2 or greater, 1.3 or greater, 1.4 or greater, or 1.5 or greater.

The viscosity of the pre-polymer of the curable composition depends in part upon the molecular weight of the pre-polymer, with higher molecular weight pre-polymers giving rise to more viscous compositions. In one application, the pre-polymer can also have a molecular weight of about 1,000 Daltons or more, about 2,000 Daltons or more, about 3,000 Daltons or more, about 4,000 Daltons or more, about 5,000 Daltons or more, about 6,000 Daltons or more, about 7,000 Daltons or more, about 8,000 Daltons or more, about 9,000 Daltons or more, about 10,000 Daltons or more, about 11,000 Daltons or more, about 12,000 Daltons or more, about 13,000 Daltons or more, about 14,000 Daltons or more, about 15,000 Daltons or more, about 16,000 Daltons or more, about 17,000 Daltons or more, about 18,000 Daltons or more, about 19,000 Daltons or more, about 20,000 Daltons or more, about 21,000 Daltons or more, about 22,000 Daltons or more, about 23,000 Daltons or more, about 24,000 Daltons or more, about 25,000 Daltons or more, about 26,000 Daltons or more, about 27,000 Daltons or more, about 28,000 Daltons or more, about 29,000 Daltons or more, about 30,000 Daltons or more, about 35,000 Daltons or more, about 40,000 Daltons or more, about 45,000 Daltons or more, about 50,000 Daltons or more, about 55,000 Daltons or more, about 60,000 Daltons or more, about 65,000 Daltons or more, about 70,000 Daltons or more, about 75,000 Daltons or more, about 80,000 Daltons or more, about 85,000 Daltons or more, about 90,000 Daltons or more, about 95,000 Daltons or more, or about 100,000 Daltons or more. The molecular weight of the pre-polymer can be provided within a range between and including any two of the foregoing values. For example, the molecular weight range can be from about 3,000 Daltons to about 10,000 Daltons.

In one application, the curable composition or adhesive comprises or consists of a sealant and the pre-polymer can have any one of the above-recited molecular weights. For example, the pre-polymer can have a molecular weight of about 11,000 Daltons or greater.

In one application, the curable composition comprises or consists of an adhesive and the pre-polymer can have any of above-recited molecular weights. For example, the pre-polymer can have a molecular weight of about 1,000 Daltons to about 10,000 Daltons.

The desired viscosity of the pre-polymer can be tuned based, in part, on the molecular weight of the pre-polymer. In one application, the desired viscosity can be selected to provide a pre-polymer that to remain in place at the site of application without being washed away by bodily fluids. The viscosity of the pre-polymer can be about 0.5 Pa·s or more, 1 Pa·s or more, 2 Pa·s or more, 3 Pa·s or more, 4 Pa·s or more, 5 Pa·s or more, 6 Pa·s or more, 7 Pa·s or more, 8 Pa·s or more, 9 Pa·s or more, 10 Pa·s or more, 11 Pa·s or more, 12 Pa·s or more, 13 Pa·s or more, 14 Pa·s or more, 15 Pa·s or more, 16 Pa·s or more, 17 Pa·s or more, 18 Pa·s or more, 19 Pa·s or more, 20 Pa·s or more, 21 Pa·s or more, 22 Pa·s or more, 23 Pa·s or more, 24 Pa·s or more, 25 Pa·s or more, 26 Pa·s or more, 27 Pa·s or more, 28 Pa·s or more, 29 Pa·s or more, 30 Pa·s or more, 31 Pa·s or more, 32 Pa·s or more, 33 Pa·s or more, 34 Pa·s or more, 35 Pa·s or more, 36 Pa·s or more, 37 Pa·s or more, 38 Pa·s or more, 39 Pa·s or more, 40 Pa·s or more, 41 Pa·s or more, 42 Pa·s or more, 43 Pa·s or more, 44 Pa·s or more, 45 Pa·s or more, 46 Pa·s or more, 47 Pa·s or more, 48 Pa·s or more, 49 Pa·s or more, 50 Pa·s or more, 51 Pa·s or more, 52 Pa·s or more, 53 Pa·s or more, 54 Pa·s or more, 55 Pa·s or more, 56 Pa·s or more, 57 Pa·s or more, 58 Pa·s or more, 59 Pa·s or more, 60 Pa·s or more, 61 Pa·s or more, 62 Pa·s or more, 63 Pa·s or more, 64 Pa·s or more, 65 Pa·s or more, 66 Pa·s or more, 67 Pa·s or more, 68 Pa·s or more, 69 Pa·s or more, 70 Pa·s or more, 71 Pa·s or more, 72 Pa·s or more, 73 Pa·s or more, 74 Pa·s or more, 75 Pa·s or more, 76 Pa·s or more, 77 Pa·s or more, 78 Pa·s or more, 79 Pa·s or more, 80 Pa·s or more, 81 Pa·s or more, 82 Pa·s or more, 83 Pa·s or more, 84 Pa·s or more, 85 Pa·s or more, 86 Pa·s or more, 87 Pa·s or more, 88 Pa·s or more, 89 Pa·s or more, 90 Pa·s or more, 91 Pa·s or more, 92 Pa·s or more, 93 Pa·s or more, 94 Pa·s or more, 95 Pa·s or more, 96 Pa·s or more, 97 Pa·s or more, 98 Pa·s or more, 99 Pa·s or more, or 100 Pa·s or more. The viscosity can be provided within a range between and including any two of the foregoing values. For example, the range for viscosity can be from about 0.5 Pa·s to about 50 Pa·s.

The pre-polymer is optionally formed by the reaction of a polyol and a polyacid. The polyol can be one or a combination of compounds comprising two or more hydroxyl groups, including diols, alkane diols, triols, glycerol, trimethylolpropane, triethanolamine, tetraols, erythritol, pentaerythritol, sorbital, unsaturated diols, tetradeca-2,12-diene-1,1,14-diol, macromonomer diols, polyethylene oxide, or N-methyldiethanolamine. The polyacid can be a diacid or higher order acid and include, for example, glutaric acid, adipic acid, pimclic acid, suberic acid, and azelaic acid. Exemplary long chain acids can include diacids having 5 or more, 10 or more, 15 or more, 20 or more, or 25 or more carbon atoms.

In one application, the pre-polymer is a poly(glycerol sebacate) (PGS) pre-polymer prepared through the polycondensation of equimolar amounts of glycerol and sebacic acid.

The curable composition can comprise an initiator. In one application, the initiator is a photoinitiator. In one application, the photoinitiator can be selected from the group consisting of 2-dimethoxy-2-phenyl-acetophenone, 2-hydroxy-1-[4-(hydroxyethoxy)phenyl]-2-methyl-1-propanone (IRGACURE® 2959), 1-hydroxycyclohexyl-1-phenyl ketone (IRGACURE® 184), 2-hydroxy-2-methyl-1-phenyl-1-propanone (DAROCUR® 1173), 2-benzyl-2-(dimethylamino)-1-[4-morpholinyl)phenyl]-1-butanone (Irgacure 369), methylbenzoylformate (DAROCUR® MBF), oxy-phenyl-acetic acid-2-[2-oxo-2-phenyl-acetoxy-ethoxy]-ethyl ester (IRGACURE® 754), 2-methyl-1-[4-(methylthio)phenyl]-2-(4-morpholinyl)-1-propanone (IRGACURE® 907), diphenyl(2,4,6-trimethylbenzoyl)-phosphine oxide (DAROCUR® TPO), phosphine oxide, phenyl bis(2,4,6-trimethyl benzoyl) (IRGACURE® 819), and combinations thereof. In one application, the preferred photoinitiator is IRGACURE® 2959.

The pre-polymer can be crosslinked by photopolymerization by exposure to electromagnetic radiation, such as visible or UV light. The exposure time can be varied in order to achieve the desired amount of crosslinking. In one application, the irradiation time is about 1 second, 5 seconds, 10 seconds, 15 seconds, 20 seconds, 30 seconds, 45 seconds, one minute, 90 seconds, or two minutes or greater. The irradiation time is provided can be in a range between and including any two values. The intensity of the light can be varied as needed to achieve sufficient crosslinking. In one application, the intensity is less than about 0.45 W/cm2.

The crosslink density in the cured polymer can be tuned by varying the degree of activation, e.g., acrylation, of the pre-polymer or by varying the curing conditions, such as cure time and the intensity of the energy that is applied to cure the pre-polymer. A greater adhesive strength is believed to be achieved by higher levels of crosslinking.

Where the resulting cross-linked polymer comprises a sealant, it can have a crosslinking density of about 10% or less, about 9% or less, about 8% or less, about 7% or less, about 6% or less, about 5% or less, about 4% or less, about 3% or less, about 2% or less, about 1% or less, about 0.5% or less, about 0.1% or less, about 0.05% or less, about 0.01% or less, about 0.005% or less, or about 0.001% or less. The resulting cross-linked polymer can have a crosslinking density within a range of between and including any two of the foregoing values.

Where the resulting cross-linked polymer comprises an adhesive, it can have a crosslinking density of about 1% or more, about 2% or more, about 3% or more, about 4% or more, about 5% or more, about 6% or more, about 7% or more, about 8% or more, about 9% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, or about 80% or more. The resulting cross-linked polymer can have a crosslinking density within a range of between and including any two of the foregoing values. The greater the crosslink density, the greater the polymer cohesion and adhesive strength.

The resulting cross-linked polymer can be configured to adhere to wet tissue. In one embodiment in which the cross-linked polymer is an adhesive, the cross-linked polymer has an adhesion strength that is sufficient to secure the implantable medical device to the anatomical feature or tissue, preferably without the need for additional securing mechanisms such as sutures or staples. Depending on the forces that can act upon the cross-linked polymer at the site of application, such as hemodynamic forces, the adhesive strength can be about 0.1 N/cm2 or greater, about 0.2 N/cm2 or greater, about 0.3 N/cm2 or greater, about 0.4 N/cm2 or greater, about 0.5 N/cm2 or greater, about 0.6 N/cm2 or greater, about 0.7 N/cm2 or greater, about 0.8 N/cm2 or greater, about 0.9 N/cm2 or greater, about 1.0 N/cm2 or greater, about 1.1 N/cm2 or greater, about 1.2 N/cm2 or greater, about 1.3 N/cm2 or greater, about 1.4 N/cm2 or greater, about 1.5 N/cm2 or greater, about 1.6 N/cm2 or greater, about 1.7 N/cm2 or greater, about 1.8 N/cm2 or greater, about 1.9 N/cm2 or greater, about 2.0 N/cm2 or greater, about 2.1 N/cm2 or greater, about 2.2 N/cm2 or greater, about 2.3 N/cm2 or greater, about 2.4 N/cm2 or greater, about 2.5 N/cm2 or greater, about 2.6 N/cm2 or greater, about 2.7 N/cm2 or greater, about 2.8 N/cm2 or greater, about 2.9 N/cm2 or greater, about 3.0 N/cm2 or greater, about 3.5 N/cm2 or greater, about 4.0 N/cm2 or greater, about 4.5 N/cm2 or greater, about 5.0 N/cm2 or greater, about 5.5 N/cm2 or greater, about 6.0 N/cm2 or greater, about 6.5 N/cm2 or greater, about 7.0 N/cm2 or greater, about 7.5 N/cm2 or greater, about 8.0 N/cm2 or greater, about 8.5 N/cm2 or greater, about 9.0 N/cm2 or greater, about 9.5 N/cm2 or greater, or about 10.0 N/cm2 or greater. The adhesion strength can be provided in a range between and including any two of the foregoing values.

Where the cross-linked polymer comprises a sealant, the cross-linked polymer can have an adhesion strength that is sufficient to permit the cross-linked polymer to remain at the site of application. In some applications, the implantable medical device can be adhered to the anatomical feature without the need for sutures or additional means for securing the device. The sealant can have the adhesive strength to secure the implantable medical device to the anatomical feature. In some applications, the sealant need only be strong enough to resist becoming dislodged from the site of application by the hemodynamic forces that can act upon it. In some applications, sutures or additional means for securing the device can optionally be used with the sealant. In one application, the adhesive strength of the sealant is about 0.1 N/cm2 or less, about 0.09 N/cm2 or less, about 0.08 N/cm2 or less, about 0.07 N/cm2 or less, about 0.06 N/cm2 or less, about 0.05 N/cm2 or less, about 0.04 N/cm2 or less, about 0.03 N/cm2 or less, about 0.02 N/cm2 or less, about 0.01 N/cm2 or less, about 0.009 N/cm2 or less, about 0.008 N/cm2 or less, about 0.007 N/cm2 or less, about 0.006 N/cm2 or less, about 0.005 N/cm2 or less, about 0.004 N/cm2 or less, about 0.003 N/cm2 or less, about 0.002 N/cm2 or less, or about 0.001 N/cm2 or less. The wet adhesion can be provided in a range between and including any two of the foregoing values.

Although the embodiments described herein relate largely to annuloplasty bands adhered to tissue of an annulus of a native heart valve, the methods, systems, and apparatuses disclosed hereinbelow are relevant to adhering the external surface of a range of implants to various tissue of a subject, mutatis mutandis.

The systems, apparatuses, and techniques described herein may be used in combination with those described in US 2018/0049875 to Iflah et al., and/or U.S. Pat. No. 9,949,828 to Sheps et al, both of which are incorporated by reference herein.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver heart, simulator (e.g. with the body parts, tissue, etc. being simulated), etc.

What is claimed is:

1. A method, comprising:
transcatheterally advancing, to a heart of a subject, an implant and an adhesive-applicator, the adhesive-applicator containing an adhesive, and the implant:
including a wall formed from a porous fabric, the wall having an external surface, and shaped to define an interior; and
while a nozzle of the adhesive-applicator is disposed within the interior, adhering the external surface of the wall to tissue of the heart by using the adhesive-applicator to apply the adhesive via the nozzle to the interior, such that a portion of the adhesive passes through the porous fabric of the wall to the external surface.

2. The method according to claim 1, further comprising contacting the external surface with the tissue prior to using the adhesive-applicator to apply the adhesive via the nozzle into the interior.

3. The method according to claim 1, further comprising, while using the adhesive-applicator to apply the adhesive via the nozzle into the interior, pressing the adhesive-applicator, in the interior, against the porous fabric.

4. The method according to claim 1, wherein using the adhesive-applicator to apply the adhesive via the nozzle into the interior comprises facing the nozzle of the adhesive-applicator flush against an interior surface of the implant.

5. The method according to claim 1, wherein using the adhesive-applicator to apply the adhesive via the nozzle into the interior comprises applying a polyethylene hydrogel into the interior.

6. The method according to claim 1, wherein using the adhesive-applicator to apply the adhesive via the nozzle into the interior comprises applying poly(glycerol sebacate acrylate) into the interior.

7. The method according to claim 1, wherein:
the implant includes a contracting portion, and a contraction member extending along at least the contracting portion, and
the method further comprises, subsequently to adhering the external surface of the wall to the tissue, contracting the contracting portion by tensioning the contraction member.

8. The method according to claim 1, wherein:
the adhesive includes a first adhesive-component, and
using the adhesive-applicator to apply the adhesive via the nozzle into the interior comprises using the adhesive-applicator to apply the first adhesive-component via the nozzle into the interior such that the first adhesive-component contacts a second adhesive-component and forms a hardened adhesive therewith.

9. The method according to claim 8, wherein using the adhesive-applicator to apply the first adhesive-component via the nozzle into the interior comprises applying an adhesive-component selected from: thrombin, fibrinogen, albumin, glutaraldehyde, gelatin-resorcinol, formaldehyde-glutaraldehyde and pentanedial-ethanedial.

10. The method according to claim 8, wherein the second adhesive-component is attached to the porous fabric, and wherein using the adhesive-applicator to apply the first adhesive-component via the nozzle into the interior comprises using the adhesive-applicator to apply the first adhesive-component via the nozzle into the interior such that the first adhesive-component contacts the second adhesive-component attached to the porous fabric and forms the hardened adhesive therewith.

11. The method according to claim 8, wherein the adhesive-applicator is a first adhesive-applicator, and wherein the method further comprises using a second adhesive-applicator to apply the second adhesive-component into the interior via a second nozzle of the second adhesive-applicator.

12. The method according to claim 11, wherein:
using the first adhesive-applicator to apply the first adhesive-component via the nozzle into the interior comprises applying at least one adhesive-component selected from the group consisting of thrombin and fibrinogen; and
using the second adhesive-applicator to apply the second adhesive-component via the second nozzle into the interior comprises applying the other adhesive-component from the group consisting of thrombin and fibrinogen.

13. The method according to claim 11, wherein:
using the first adhesive-applicator to apply the first adhesive-component via the nozzle into the interior comprises applying at least one adhesive-component selected from the group consisting of albumin and glutaraldehyde; and
using the second adhesive-applicator to apply the second adhesive-component via the second nozzle into the interior comprises applying the other adhesive-component from the group consisting of albumin and glutaraldehyde.

14. The method according to claim 11, wherein:
using the first adhesive-applicator to apply the first adhesive-component via the nozzle into the interior comprises applying at least one adhesive-component selected from the group consisting of gelatin-resorcinol and formaldehyde-glutaraldehyde; and
using the second adhesive-applicator to apply the second adhesive-component via the second nozzle into the interior comprises applying the other adhesive-component from the group consisting of gelatin-resorcinol and formaldehyde-glutaraldehyde.

15. The method according to claim 11, wherein:
using the first adhesive-applicator to apply the first adhesive-component via the nozzle into the interior comprises applying at least one adhesive-component selected from the group consisting of gelatin-resorcinol and pentanedial-ethanedial; and
using the second adhesive-applicator to apply the second adhesive-component via the second nozzle into the interior comprises applying the other adhesive-component from the group consisting of gelatin-resorcinol and pentanedial-ethanedial.

16. The method according to claim 1, further comprising curing the adhesive by applying energy to the adhesive from an adhesive-curing device.

17. The method according to claim 16, wherein applying energy to the adhesive from the adhesive-curing device comprises applying heat to the adhesive from the adhesive-curing device.

18. The method according to claim 16, wherein applying energy to the adhesive from the adhesive-curing device comprises applying ultraviolet radiation to the adhesive from the adhesive-curing device.

19. The method according to claim 1, wherein:
adhering the external surface to the tissue comprises adhering a first part of the external surface to the tissue,
the method further comprises, subsequently to adhering the first part of the external surface to the tissue:
moving the nozzle to an other location within the interior, and
while the nozzle is disposed in the other location within the interior, adhering a second part of the external surface of the wall to the tissue by using the adhesive-applicator to apply the adhesive via the nozzle to the interior, such that another portion of the adhesive passes through the porous fabric to the second part of the external surface.

20. The method according to claim 19, wherein:
adhering the first part of the external surface to the tissue comprises adhering the first part of the external surface to tissue at a left fibrous trigone of the heart, and
adhering the second part of the external surface to the tissue comprises adhering the second part of the external surface to tissue at a right fibrous trigone of the heart.
